(19) United States
(12) Patent Application Publication
(10) Pub. No.: US 2021/0220575 A1
(43) Pub. Date: Jul. 22, 2021

(54) INHALATION DEVICE WITH A PUMPING UNIT

(71) Applicant: Softhale NV, Diepenbeek (BE)

(72) Inventors: Frank Bartels, Hattingen (DE); Juergen Rawert, Koeln (DE)

(21) Appl. No.: 17/055,612

(22) PCT Filed: May 28, 2019
(86) PCT No.: PCT/EP2019/063768

§ 371 Date: Nov. 16, 2020

Related U.S. Application Data
(60) Provisional application No. 62/677,846, filed on May 30, 2018.

(30) Foreign Application Priority Data
May 30, 2018 (EP) 18175048.0

(51) Int. Cl. A61M 11/00; B05B 11/00; A61M 11/02

(52) U.S. Cl. CPC A61M 11/007; B05B 11/3004; B05B 11/3091; B05B 11/305; A61M 11/02

(57) ABSTRACT

The invention relates to the field of inhalation devices for liquids. In particular, the invention relates to an inhalation device for delivering a nebulised medically active aerosol for inhalation therapy comprising a pumping unit, said pumping unit comprising a riser pipe (2), a hollow cylindrical part (3) having an interior space configured to receive an upstream end portion (21) of said riser pipe (2), said cylindrical part (3) being moveable on the riser pipe (2), wherein the cylindrical part and the riser pipe form a pumping chamber having a variable volume, wherein a gap (4) is present between a lateral outside surface (22) of the upstream end portion (21) of said riser pipe (2) and a lateral internal surface (31) of said cylindrical part (3), wherein a seal (1) is present between the outside surface (22) of said riser pipe (2) and the internal surface (31) of said cylindrical part (3), thus physically bridging said gap (4), and wherein the seal (1) has a cap-like shape configured to fit around the upstream end portion (21) of the riser pipe (2), having at least one opening (11) for providing a fluid connection between an interior space of said riser pipe (2) and said pumping chamber, and wherein at an interior surface (14) of the seal (1), at least one interior protrusion (16) is present which is configured to be received by at least one respective recess (23) of the riser pipe (2).

### FIELD OF THE INVENTION

The invention relates to the field of inhalation devices for liquids. In particular, the invention relates to a pumping unit for an inhalation device having a piston which moves relative to a pumping chamber in order to generate pumping pressure.

### BACKGROUND OF THE INVENTION

Nebulizers or other aerosol generators for liquids are known from the art since a long time ago. Amongst others, such devices are used in medical science and therapy. There, they serve as inhalation devices for the application of active ingredients in the form of aerosols, i.e. small liquid droplets embedded in a gas. Such an inhalation device is known e.g. from document EP 0 627 230 B1. Essential components of this inhalation device are a reservoir in which the liquid that is to be aerosolized is contained; a pumping unit for generation of a pressure being sufficiently high for nebulizing; as well as an atomizing device in the form of a nozzle. A pumping unit is defined as a unit, device or component capable of moving or compressing a fluid material and that comprises at least one pumping chamber, and optionally further comprises auxiliary components as well, such as a body, interfaces, and the like. By means of the pumping unit, the liquid is drawn in a discrete amount, i.e. not continuously, from the reservoir, and fed to the nozzle. The pumping unit works without propellant and generates pressure mechanically.

This inhalation device makes use of a fixed pumping chamber, into which a moveable and hollow piston can be inserted in order to decrease the interior volume of said chamber, thus increasing the pressure both in said chamber and the inside of the piston, eventually leading to atomization of the liquid from the nozzle. By again extracting the piston from the chamber, its interior volume is increased, and the resulting negative pressure leads to drawing liquid from the reservoir into the chamber, such that a new atomizing cycle can begin.

An improvement of such an inhalation device is disclosed in patent application EP 17168869.0, filed by the same applicant as the present invention, the content of which is incorporated herein in its entirety. According to this improvement, the inhalation device provides a fixed piston and a moveable pumping chamber with a hollow cylindrical part.

By pushing the cylindrical part onto the hollow piston, the pressure inside the pumping chamber is increased and the liquid is pressed out of the nozzle. By pulling the cylindrical part from the piston, the pressure becomes negative and fresh liquid is drawn into the increasing volume of the chamber.

An improvement which is directed in particular to the problem of sealing the gap between immobile piston and moveable pumping chamber of such an inhalation device is disclosed in application EP 17197136.9 which is also filed by the same applicant as the present invention, the content of which is also incorporated herein in its entirety. According to this improvement, the seal which is bridging said gap in order to minimize pressure loss when a pressure is generated inside said pumping chamber is characterized in that said seal is fixed to said outside of said riser pipe. However, it was experienced that, due to the particularly high pressure it can be exposed to, the seal is prone to fatigue and seat problems.

As a result, the pumping unit which comprises piston, pumping chamber and seal is prone to such failure as well.

U.S. Pat. No. 4,067,499 discloses a specific non-aerosol type spray device comprising a container for housing a liquid content material to be dispensed and a plunger slidable as a piston within the container.

### OBJECT OF THE INVENTION

The object of the invention is the provision of a pumping unit that avoids the drawbacks of the known art. The pumping unit's seal shall provide better fatigue resistance and avoid seat problems, and the pumping unit shall as well have an increased long-term stability.

### SUMMARY OF THE INVENTION

In a first aspect, the present invention provides an inhalation device for delivering a nebulised medically active aerosol for inhalation therapy comprising a pumping unit, said pumping unit comprising a riser pipe (2), a hollow cylindrical part (3) having an interior space configured to receive an upstream end portion (21) of said riser pipe (2), said cylindrical part (3) being moveable on the riser pipe (2), wherein the cylindrical part and the riser pipe form a pumping chamber having a variable volume, wherein a gap (4) is present between a lateral outside surface (22) of the upstream end portion (21) of said riser pipe (2) and a lateral internal surface (31) of said cylindrical part (3), wherein a seal (1) is present between the outside surface (22) of said riser pipe (2) and the internal surface (31) of said cylindrical part (3), thus physically bridging said gap (4), and wherein the seal (1) has a cap-like shape configured to fit around the upstream end portion (21) of the riser pipe (2), having at least one opening (11) for providing a fluid connection between an interior space of said riser pipe (2) and said pumping chamber, and wherein at an interior surface (14) of the seal (1), at least one interior protrusion (16) is present which is configured to be received by at least one respective recess (23) of the riser pipe (2).

In a second aspect, the present invention provides a pumping unit for delivering a nebulised medically active aerosol for inhalation therapy, said unit comprising a riser pipe (2), a hollow cylindrical part (3) having an interior space configured to receive an upstream end portion (21) of said riser pipe (2), said cylindrical part (3) being moveable on the riser pipe (2), wherein the cylindrical part and the riser pipe form a pumping chamber having a variable volume, wherein a gap (4) is present between a lateral outside surface (22) of the upstream end portion (21) of said riser pipe (2) and a lateral internal surface (31) of said cylindrical part (3), wherein a seal (1) is present between the outside surface (22) of said riser pipe (2) and the internal surface (31) of said cylindrical part (3), thus physically bridging said gap (4), wherein the seal (1) has a cap-like shape configured to fit around the upstream end portion (21) of the riser pipe (2), having at least one opening (11) for providing a fluid connection between an interior space of said riser pipe (2) and said pumping chamber, and wherein at an interior surface (14) of the seal (1), at least one interior protrusion (16) is present which is configured to be received by at least one respective recess (23) of the riser pipe (2).

### BRIEF DESCRIPTION OF DRAWINGS

FIG. 1 shows a three-dimensional perspective view of a specific embodiment of the seal of the pumping unit of the inhalation device according to the invention;

FIG. 2 shows a two-dimensional cut view of the key functional part of a preferred pumping unit with the seal from FIG. 1;

FIG. 3 shows a two-dimensional cut view of the key functional part of a pumping unit of the inhalation device according to the invention with a simplified embodiment of a seal.

### DETAILED DESCRIPTION OF THE INVENTION

The object is solved by an inhalation device for delivering a nebulised medically active aerosol for inhalation therapy comprising a pumping unit, said pumping unit comprising a riser pipe (2), a hollow cylindrical part (3) having an interior space configured to receive an upstream end portion (21) of said riser pipe (2), said cylindrical part (3) being moveable on the riser pipe (2), wherein the cylindrical part and the riser pipe form a pumping chamber having a variable volume, wherein a gap (4) is present between a lateral outside surface (22) of the upstream end portion (21) of said riser pipe (2) and a lateral internal surface (31) of said cylindrical part (3), wherein a seal (1) is present between the outside surface (22) of said riser pipe (2) and the internal surface (31) of said cylindrical part (3), thus physically bridging said gap (4), and wherein the seal (1) has a cap-like shape configured to fit around the upstream end portion (21) of the riser pipe (2), having at least one opening (11) for providing a fluid connection between an interior space of said riser pipe (2) and said pumping chamber, and wherein at an interior surface (14) of the seal (1), at least one interior protrusion (16) is present which is configured to be received by at least one respective recess (23) of the riser pipe (2).

Advantageous embodiments of the inhalation device as well as of the pumping unit of the present invention are described in the dependent claims, the subsequent description, as well as the accompanying figures.

The pumping unit serves for the inhalation device of the present invention for delivering a nebulised medically active aerosol for inhalation therapy.

In specific embodiments, the inhalation device according to the invention comprises a housing, inside this housing a reservoir for storing a liquid, and a pumping unit which is preferably based on the principle of a piston pump, or plunger pump. More specifically, said pumping unit according to the invention comprises a hollow cylindrical part having an interior space for generation of a pressure inside a pumping chamber. It is noted that the term “cylindrical part” refers to a part having a cylindrical internal surface; the outside as well as a portion which does not come in contact with the riser pipe and/or the seal do not have to be cylindrical. (In fact, if the riser pipe has another than a cylindrical outer surface, such as e.g. a square cross section, all portions of interacting parts should match this shape.) The pumping chamber is formed by the cylindrical part and a riser pipe and has a variable volume. The pumping chamber is fluidically connected with the reservoir; optionally, via a check valve which blocks in direction of the reservoir. To achieve the volume variability, said riser pipe can be received with at least one reservoir-facing, interior end (subsequently called “upstream end”) in said cylindrical part, and the cylindrical part is moveable on the riser pipe. A nozzle is connected liquid-tight to a downstream, exterior end of the riser pipe.

In specific embodiments, the nozzle connected to the downstream, exterior end of the riser pipe may be a multi-beam collision nozzle. Such nozzles are characterized in that they comprise a multitude of, specifically at least two, such as two to about five or four, or more specifically two liquid channels from which liquid is emitted at a high velocity. Specifically, the individual jets run along respective ejection trajectories and intersect with one another, thereby forming a fine mist at the collision point.

The riser pipe of the pumping unit can be immobile with respect to the housing of the device, or at least to a part of the housing to which also the nozzle is firmly affixed, directly or indirectly, so that the riser pipe is also immobile or unmoveable relative to the nozzle. In another embodiment, the riser pipe is moveable, and the pumping chamber/cylindrical part is immobile with respect to said housing.

The interior volume of the pumping chamber is changeable by means of relative motion of the cylindrical part to the riser pipe. This means that either the cylindrical part of the pumping chamber is moveable and the riser pipe is fixed, or the cylindrical part of the pumping chamber is fixed and the riser pipe is moveable, or both cylindrical part and riser pipe are moveable. In other words, the cylindrical part has an interior space configured to receive an upstream end portion of said riser pipe. The cylindrical part is configured to be moveable along said end portion such that the volume inside said cylindrical part is changeable by means of relative motion of said riser pipe or vice versa.

A gap is present between a lateral outside surface of the upstream end portion of said riser pipe and a lateral internal surface of said cylindrical part, and a seal physically bridges said gap in order to minimize pressure loss when a pressure is generated inside said pumping chamber.

According to the invention, the seal has a cap- or pot-like shape configured to fit around the end portion of the riser pipe. This means that the seal has a bottom portion which is configured to match the respective upstream end surface of the riser pipe, and that it has an interior surface portion which is configured to match the respective outside surface end portion of the riser pipe. In order to let liquid pass through the seal's upstream end, the seal has at least one opening. In other words, the opening provides a fluid connection between the interior space of the riser pipe and the pumping chamber. This opening can be a central opening, being in line with the longitudinal axis of the riser pipe. However, also a plurality of openings can be provided, serving e.g. also as filter structures; these openings can be present in the “bottom” of the seal as an axial opening, and/or in the circumference as radial openings.

Further, at an interior surface of the seal, at least one interior protrusion is present which is configured to be received by at least one respective recess of the riser pipe. In one of the preferred embodiments, the protrusion is annular or circumferential, and the corresponding recess in the upstream end portion of the riser pipe is an also annular recess and configured to match the shape of the protrusion.

Such a seal can be firmly attached to the upstream end of a riser pipe since due to the cap-like shape, since it has a significantly increased surface that can interact with the pipe. Also, the seal will not be able to slide in downstream direction along the outside wall of the pipe, since the bottom portion inhibits such movement. This is particularly the case during the high-pressure phase when the interior space of the pumping chamber is reduced and liquid is forced through the riser pipe and through the nozzle. Further, the feature of the interior protrusion and recess provide a mechanical form-fitting interaction of both parts so that the seal stays fixed to the riser pipe not only during the pressure phase, but also the suction phase, when the pressure inside the pressure chamber becomes smaller than ambient pressure, which otherwise could lead to pulling the seal off its seat on the pipe.

The seal may be made of a broad variety of synthetic or natural elastomeric materials. In specific embodiments, these elastomeric materials may, for example, comprise synthetic rubbers such as fluoropolymeric elastomers, e.g. Viton®, nitrile butadiene rubber (NBR) or ethylene propylene diene monomer rubber (EPDM).

According to a specific embodiment of the invention, the seal has at its exterior surface a circumferential, or annular, exterior protrusion. The purpose of exterior protrusion is to provide a structure that physically bridges the gap. In this way, not the entire exterior lateral surface of the seal comes in physical contact with the internal surface of the cylindrical part, but only a precisely controllable region which can be tailored to provide optimal mechanical and physical properties, as will be shown hereinbelow.

According to a specific embodiment of said exterior protrusion, the seal has at the position of the exterior protrusion, viewed in upstream direction along a longitudinal axis, a cross section which has a diameter-increasing (or thickness-increasing) portion, followed by a diameter-reducing (or thickness-reducing) portion. This allows for facilitated insertion of the seal with pipe into the cylindrical part, and an advantageous development of physical contact is provided.

In one embodiment, the riser pipe has a nominal outer diameter, and the riser pipe's upstream end portion has a reduced diameter. This allows to have an increased wall thickness of the seal, while not increasing the width of the gap in order to provide suitable space for the seal. An increased service life of the seal can thus be achieved.

In one embodiment wherein the seal has said annular exterior protrusion as defined above, only the region of the seal where the exterior protrusion is present exceeds, in a non-assembled state in which it is not deformed, the nominal outer diameter of the riser pipe. The advantage of said embodiment is that there is only one clearly definable feature, namely the exterior protrusion, which is responsible for the sealing effect.

The term ‘liquid’ as referred to herein may be a fluid material capable of altering its shape to that of a container which holds the liquid but retains a nearly constant volume independent of pressure. A liquid may represent a monophasic liquid solution or a dispersion with a continuous liquid phase and a dispersed phase which may or may not be liquid. More specifically, the ‘liquid’ or ‘medically active liquid’ as referred to herein may be transformed to a ‘medically active aerosol for inhalation therapy’ to be administered by the inhalation device according to the present invention by nebulization or aerosolization.

In specific embodiments, the term liquid as used herein refers to a medically active liquid, more specifically a medically active liquid in form of a pharmaceutical composition comprising at least one active pharmaceutical ingredient (API). More specifically, such at least one inhalable active pharmaceutical ingredient may, for example, be selected from long-acting muscarinic antagonists (LAMA), long-acting beta agonists (LABA) and inhalable glucocorticoids (ICS), as well as from analgetics and antidiabetics, either alone or in combination which each other.

Examples for long-acting muscarinic antagonists (LAMA) comprise, but are not limited to aclidinium bromide, glycopyrronium salts, such as glycopyrronium bromide, revefenacin, tiotropium, such as tiotropium bromide, umeclidinium bromide, oxitropium bromide, flutropium bromide, ipratropium bromide, trospium chloride, tolterodine.

Examples for long-acting beta agonists (LABA) comprise, but are not limited to, albuterol, arformoterol, bambuterol, bitolterol, broxaterol, carbuterol, clenbuterol, fenoterol, formoterol, hexoprenaline, ibuterol, indacaterol, indacterol, isoetharine, isoprenaline levosalbutamol, mabuterol meluadrine, metaproterenol, olodaterol, orciprenaline, pirbuterol, procaterol, reproterol, rimiterol, ritodrine, salmeterol, salmefamol, soterenot, sulphonterol, tiaramde, terbutaline, terbuterol.

Examples of inhalable glucocorticoids (ICS) comprise, but are not limited to, prednisolone, prednisone, butixocort propionate, flunisolide, beclomethasone, triamcinolone, budesonide, fluticasone, mometasone, ciclesonide, rofleponide, dexamethasone, etiprednol-dichloroacetat, deflazacort, etiprednol, loteprednol, RPR-106541, NS-126, ST-26.

Furthermore, active pharmaceutical ingredients may be selected from analgetics, such as opioid analgetics (e.g. morphine, fentanyl) or non-opioid analgetics (e.g. salicylic acid derivates, e.g. acetylsalicylic acid) or cannabinoids (e.g. tetrahydrocannabinol), antidiabetics, such as insulin.

The medically active liquid or liquid pharmaceutical composition to be nebulized or aerosolized by the present inhalation device may comprise at least one active pharmaceutically ingredient as described above, but may also comprise a mixture of two or more active pharmaceutically ingredients that may be administered by inhalation.

The medically active liquid or pharmaceutical composition that may be administered by the inhalation device according to the invention is preferably formulated as a composition that is suitable, and adapted for inhalative use, in other words a composition that may be nebulized or atomized for inhalation and that is physiologically acceptable for inhalation by a subject.

The medically active liquid or pharmaceutical composition that may be administered by the inhalation device according to this aspect of the invention or contained within the inhalation device and reservoir may be in the form of a dispersion, for example a suspension with a liquid continuous phase, and a solid dispersed phase or in the form of a solution.

In further embodiments, the medically active liquid or pharmaceutical composition as described above may comprise, optionally, one or more physiologically acceptable excipients, which are suitable for inhalative use. Excipients which may be featured in the composition may include, but are not limited to, one or more buffering agents to regulate or control pH of the solution, salts, taste-masking agents, surfactants, lipids, antioxidants, and co-solvents, which may be used to enhance or improve solubility, for example ethanol, or a glycol.

In specific embodiments, the liquids or liquid compositions as described above may be essentially free of a propellant.

In further specific embodiments, the liquids or liquid compositions as described above may be an aqueous solution, in which one or more active pharmaceutical ingredients as described above are dissolved and solubilized in a liquid carrier solution comprising water. Such aqueous solutions optionally may also comprise one or more excipients as described above.

In a second aspect, the present invention provides a pumping unit for delivering a nebulised medically active aerosol for inhalation therapy, said unit comprising a riser pipe (2), a hollow cylindrical part (3) having an interior space configured to receive an upstream end portion (21) of said riser pipe (2), said cylindrical part (3) being moveable on the riser pipe (2), wherein the cylindrical part and the riser pipe form a pumping chamber having a variable volume, wherein a gap (4) is present between a lateral outside surface (22) of the upstream end portion (21) of said riser pipe (2) and a lateral internal surface (31) of said cylindrical part (3), wherein a seal (1) is present between the outside surface (22) of said riser pipe (2) and the internal surface (31) of said cylindrical part (3), thus physically bridging said gap (4), wherein that the seal (1) has a cap-like shape configured to fit around the upstream end portion (21) of the riser pipe (2), having at least one opening (11) for providing a fluid connection between an interior space of said riser pipe (2) and said pumping chamber, and wherein at an interior surface (14) of the seal (1), at least one interior protrusion (16) is present which is configured to be received by at least one respective recess (23) of the riser pipe (2).

For the avoidance of doubt, all embodiments including all specific embodiments as described above in connection with the inhalation device according to the first aspect of the present invention, as well as all combinations thereof, may refer to the pumping unit according to this second aspect of the invention, as well.

The invention will be exemplarily described by aid of the following figures:

### DETAILED DESCRIPTION OF DRAWINGS

FIG. 1 shows a three-dimensional perspective view of a specific embodiment of the seal of the pumping unit of the inhalation device according to the invention;

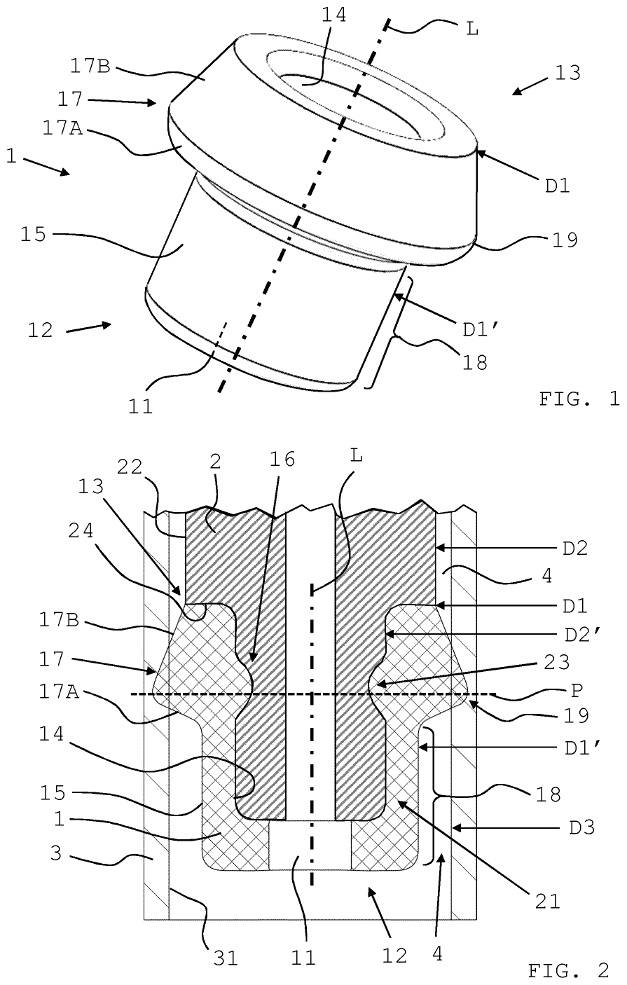

FIG. 2 shows a two-dimensional cut view of the key functional part of a preferred pumping unit with the seal from FIG. 1;

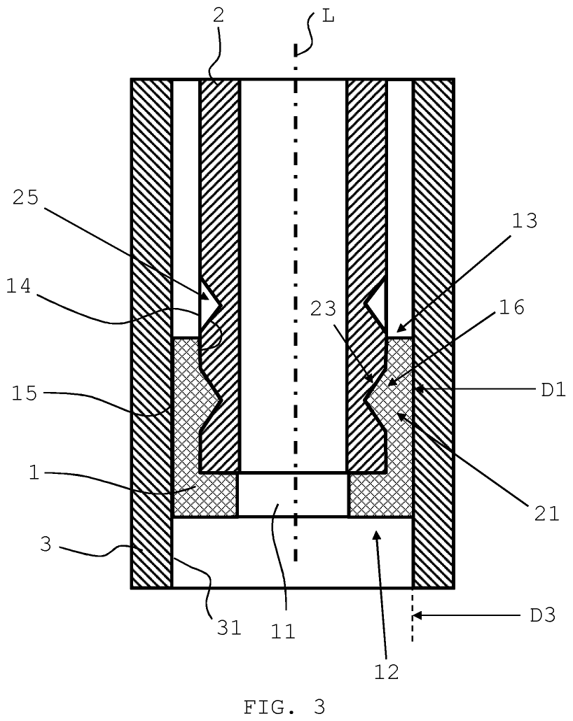

FIG. 3 shows a two-dimensional cut view of the key functional part of a pumping unit of the inhalation device according to the invention with a simplified embodiment of a seal.

In FIG. 1, a three-dimensional perspective view of a preferred embodiment of the seal 1 for incorporation in a pumping unit that may serve for an inhalation device according to the invention is shown. The seal 1 has a rotation symmetric shape (rotational axis corresponds to longitudinal axis L), with an upstream end 12 and a downstream end 13. During operation, liquid, more specifically a liquid pharmaceutical composition comprising at least one active pharmaceutical ingredient, (not shown) will flow from the upstream end 12 to the downstream end 13. One opening 11 (hidden in FIG. 1) is present in the upstream end 12 of the seal 1, to let liquid enter and pass through the seal 1. As can be seen, seal 1 has a pot- or cap-like shape, with the “pot bottom” at the upstream end 12, and the “pot opening” (not to be confused with aforesaid opening 11) at the upstream end 13, at the top of the picture.

More details can be seen in FIG. 2, which shows a two-dimensional cut view of the major functional parts of a pumping unit with the seal from FIG. 1, namely a cylindrical part 3 enclosing a pumping chamber, and the upstream end of a riser pipe 2.

As can be seen in this Figure, at an interior surface 14 of the seal 1, a protrusion 16 is present. Said protrusion 16 is configured to interact with the outside surface 22 of the riser pipe 2. To achieve this, protrusion 16 is configured to be received by a respective recess 23 of the riser pipe 2. The object of protrusion 16 with recess 23 is to inhibit slippage and relative motion between seal 1 and riser pipe 2.

According to one embodiment of the pumping unit, the at least one protrusion 16 of seal 1 is present as an annularly arranged protrusion. That means that the protrusion is a circumferential and continuous portion of seal 1.

According to another embodiment, the annularly arranged interior protrusion is designed as a discontinuous protrusion. This is the case when an annular single protrusion has one or more interruptions, which would restrict a rotational relative movement of the seal on the pipe. Similarly, a plurality of individual dimples may be provided as protrusions, or the protrusion may be provided by a nap structure. It is clear that the corresponding recess 23 of the riser pipe 2 should be shaped or configured to sufficiently match the shape of the protrusion(s) in order to fulfill the desired function. However, it is possible to provide more recesses than protrusions, as long as every protrusion can be received by one recess. This can be advantageous ifs single design of a riser pipe should be provided for several types of seals, i.e. in order to match different pressures, gap widths, etc.

Preferably, the interior protrusion 16 is spaced apart from the upstream end 12 as well as the downstream end 13 of the seal 1. As a result, compared to a position of the interior protrusion 16 being close to, or directly in contact with downstream end 13, the non-slippage effect is even increased. Since the extensibility of seal 1 at the preferred location is lower, interior protrusion 16 sits more tightly in recess 23. At the same time, downstream end 13 of seal 1 can be designed with a decreased wall thickness, and the wall thickness of the upstream end portion 21 of riser pipe 2 can be increased.

According to a particularly preferred embodiment, in a cross section along the longitudinal axis L, the contour of the protrusion 16 has a concave shape towards the riser pipe. This means that the surface which extends towards the riser pipe 2 is similar to a section of a torus. The advantage of said shape is that assembling the seal 1 with the riser pipe 2 is facilitated, but still preserving the intended non-slippage effect. Further, the matching negative (concave) shape which is preferably present in the riser pipe 2 is easy to fabricate and imposes minimum negative mechanical effects (notching effect) on the end portion 21 of pipe 2.

In another embodiment, the contour of the interior protrusion has a polygonal shape, with or without undercut. A polygonal shape can be e.g. rectangular, edged, or tapered. An additional undercut serves as a “jamming means”, allowing the seal to establish a very tight and firm connection once it is in place. Since the seal is elastic, it can nevertheless be mounted as desired onto the matching portion of the pipe. It is clear that preferably, that pipe region provides a matching cross section which is substantially a negative of the interior protrusion.

As mentioned above, preferably, the seal 1 has at its exterior surface (15) an annular exterior protrusion 17. It should be mentioned that in FIG. 2, for the sake of clarity, the exterior protrusion 17 is shown in a state prior to insertion into the pumping chamber 3, i.e. with an external diameter exceeding the internal diameter of the cylindrical part 3. It is clear that in the assembled state, due to its resilience or elasticity, exterior protrusion 17 will be deformed to fit with the cylindrical part and to be in contact with the internal surface 31. It is also clear that the exterior protrusion 17 must be circumferential in order to completely close the gap 4. However, exterior protrusion 17 can be designed to have always the same distance to the upstream end 12 (as a flat ring), but it also can have, along its circumference, varying distances (e.g. as a “wave-shaped” ring), as long as the protrusion 17 is not interrupted.

According to the depicted embodiment of said exterior protrusion 17, the seal 1 has at the position of the exterior protrusion 17, viewed in upstream direction along a longitudinal axis L, a cross section which has a diameter-increasing (or thickness-increasing) portion 17A, followed by a diameter-reducing (or thickness-reducing) portion 17B. These portions 17A, 17B can be seen both in FIGS. 1 and 2. The advantage of such portions 17A, 17B is that inserting the seal 1 which is attached to the pipe 2 into the cylindrical part 3 which encloses the pumping chamber is facilitated. Further, the increasing-decreasing contour allows for an advantageous development of physical contact between the seal 1 and the internal surface 31 of the cylindrical part 3, which will be shown below. When inserted into the cylindrical part 3, the shape of portions 17A, 17B will be accordingly deformed (concavely curved, not shown), further improving the sealing effect.

Preferably, in a cross-sectional view along the longitudinal axis L, both portions 17A, 17B have sections with linear contours, as can be seen in particular in FIG. 2. However, a rounded transition between these two sections is preferred, too.

Also, the angle between the contours of the two sections of said portions 17A, 17B is preferably a right angle. This results in an easy to fabricate geometry that still provides the aforementioned positive sealing effects.

For example, the angle of the linear section of the diameter increasing portion 17A may be about 20 degrees±10 degrees with respect to the longitudinal axis L. This value provides particularly good assembly as well as sealing properties.

According to another embodiment, a region 18 upstream of the exterior protrusion 17 exists where the seal 1 has a diameter D1′ which is smaller than the nominal diameter D1 which is located, in the present example, at the downstream end 13 of the seal 1. This also can be seen in FIG. 2. As a result, gap 4 is wider at the upstream end 12 than it is at the downstream end 13 of the seal. As can also be seen in FIG. 2, this allows, on one hand, for a reinforced downstream end 13 close to which the exterior protrusion 17 can be positioned, while, on the other hand, reducing the necessary material at the upstream end 12 portions of the seal 1.

Reduced diameter D1′ can preferably have a value which is 0.1, 0.15, 0.2, 0.25, 0.3, 0.4, 0.5, 0.6, 0.8, or 1.0 mm smaller than nominal diameter D1.

According to a preferred embodiment, the largest diameter 19 of the exterior protrusion 17 lies within a plane P which is perpendicular to the longitudinal axis L, and which crosses the protrusion 16. In other words, in this embodiment, a plane P exists which (i) is perpendicular to the longitudinal axis L and (ii) which coincides with said largest diameter 19 of the externally arranged exterior protrusion 17, and said plane P also (iii) intersects with any region of the internally arranged interior protrusion 16. In FIG. 2, this plane P is indicated by the dashed horizontal line. The advantage is that the forces that act during operation of the pumping unit onto seal 1, and particularly onto exterior protrusion 17, compressing the same towards riser pipe 2, lead also to pressing the interior protrusion 16 into the respective matching recess 23 of pipe 2 (see below). Thus, a particularly tight seal and form fit is provided. This would not be the case if the axial positions of interior protrusion 16 and exterior protrusion 17 would be different, and not (at least approximately) the same.

As mentioned above in a preferred embodiment which is also visible in FIG. 2, the riser pipe 2 has a nominal outer diameter D2, and the riser pipe's 2 upstream end portion 21 has a reduced diameter D2′. This allows to have an increased wall thickness of the seal 1, while not increasing the width of gap 4 in order to provide suitable space for the seal. An increased service life of seal 1 can thus be achieved.

Also, in this way, a “shoulder” 24 is provided against which the downstream end 13 of seal 1 can rest; this is particularly advantageous during the pressure phase of the pumping action. Thus, shoulder 24 complements the fixing function of the cap-shaped upstream end 12.

Values for the nominal outer diameter D2 are 1.0, 1.5, 2.0, 2.5, 3, 4, and 5 mm. Reduced diameter D2′ can preferably have a value which is 0.1, 0.15, 0.2, 0.25, 0.3, 0.4, 0.5, 0.6, 0.8, or 1.0 mm smaller than nominal outer diameter D2.

In one embodiment wherein the seal 1 has said annular exterior protrusion 17 as defined above, only the region of the seal 1 where the exterior protrusion 17 is present exceeds, in the non-assembled state in which it is not deformed, the nominal outer diameter D2 of riser pipe 2. This means that the nominal diameter D1 of the seal 1, located exactly at its downstream end 12 in the example of FIG. 2, does not exceed the nominal outer diameter D2 of the riser pipe 2. In the example shown in FIG. 2, obviously, also reduced diameter D1′ of upstream end 12 of seal 1 does not exceed nominal outer diameter D2. Only the exterior protrusion 17 itself extends from said diameters D1, D2, which is necessary since the exterior protrusion 17 must bridge gap 4 in order to provide a physical contact with the internal surface 31 of the cylindrical part 3.

As mentioned above, the advantage of said embodiment is that there is only one clearly definable feature, namely exterior protrusion 17, which is responsible for the sealing effect. Thus, the sealing function is easier to control. Also, only a reduced amount of material comes in physical contact with the internal surface 31 of cylindrical part 3, reducing friction, and providing more space for elastic deformation of the contacting regions of seal 1.

According to one embodiment, the riser pipe 2 has a nominal outer diameter D2, and the diameter at the downstream end 13 of the seal 1 matches said nominal outer diameter D2 of the pipe 2. This results in a smooth transition from the downstream “edge” of seal 1 to the shell surface of riser pipe 2, also facilitating assembly of both parts with the cylindrical part 3. In the present example of FIG. 2, nominal diameter D1 is located exactly at downstream end 13; however, nominal diameter D1 could also cover also an upstream region of certain axial length measured from this end.

In one embodiment, the nominal diameter D1 is slightly (0.01 to 0.5 mm) larger than the aforementioned “transition diameter” at the downstream end 13, but still smaller than the largest diameter 19 at the exterior protrusion 17.

In a preferred embodiment, in a non-assembled state, the largest diameter 19 of exterior protrusion 17 exceeds the inner diameter D3 of the cylindrical part 3, such as to be able to physically close the gap 4 between riser pipe 2 and pumping chamber 3 in an assembled state. It is clear that some physical contact must be established in order to get the seal 1 according to the invention to work properly; preferably, this contact is established by the exterior protrusion 17 alone. Such a construction results in a friction based sealing of the gap 4. Inner diameter D3 typically ranges between 0.5 and 10.0 mm. Gap 4 typically ranges from 0.005 to 1.0 mm.

In another embodiment, the largest diameter 19 does not exceed inner diameter D3 of cylindrical part 3 when only ambient pressure is present. In this case, only the high pressure inside the chamber results in an initial deformation of seal 1, which only then physically closes gap 4. If properly designed, the amount of liquid which passes gap 4 prior to the pressure-induced sealing effect coming into force is extremely low. However, this low amount can advantageously be used for flushing particles out of the gap. Such particles which can be the result of friction and abrasion as well as be components of the liquid itself could otherwise inhibit proper functioning of the seal.

In order to obtain a long lasting, failure proof design, the shore hardness of the seal 1 must be adjusted (i) to the pressure that the pumping chamber 3 can be exposed to, and (ii) to the width of the gap 4, such that, when seal 1 is exposed to said pressure, a tight seal is provided, while elastic deformation of the same is allowed, but creeping of the exterior protrusion 17 is prohibited. If the parameters are not adjusted to one another, the exterior protrusion 17 will either creep, resulting is quick fatigue and failure of the seal, or too much friction would require too much force to move the pipe relative to the cylindrical part. A too small and/or too soft exterior protrusion 17 would not seal well at high pressures, and also wear off too quick.

FIG. 3, wherein some of the already introduced reference numerals are omitted, shows an example of a more simple embodiment of the seal 1 together with riser pipe 2 and cylindrical part 3. In this embodiment, the entire outside surface 15 of seal 1 serves as the sealing surface which makes physical contact with the internal surface 31 of cylindrical part 3. A specific exterior protrusion is not present, or, from an alternative point of view, the entire outside surface 15 serves as external protrusion.

In this embodiment as well, in a disassembled state, the nominal diameter D1 of seal 1 extends beyond inner diameter D3 of cylindrical part 3. This results in the assembled state in physical contact between seal 1 and cylindrical part 3, as depicted in FIG. 3.

As can also be seen in FIG. 3, interior protrusion 16 as well as matching recess 23 have not a concave shape, but a tapered shape.

Further, riser pipe 2 has a second recess 25 positioned axially downstream to the first recess 16. Thus, a longer seal 1 could be used without having to alter the upstream end portion 21 of riser pipe 2.

### LIST OF REFERENCES

1 seal
11 opening
12 upstream end
13 downstream end
14 interior surface
15 exterior surface
16 interior protrusion
17 exterior protrusion
17A diameter increasing portion, portion
17B diameter reducing portion, portion
18 region
19 largest diameter
2 riser pipe, pipe
21 upstream end portion, end portion
22 outside surface
23 recess
24 shoulder
25 second recess
3 cylindrical part
31 internal surface
4 gap
L longitudinal axis
P plane
D1 nominal diameter (of the seal)
D1′ diameter (of the downstream end)
D2 nominal outer diameter (of the riser pipe)
D2′ reduced diameter
D3 inner diameter (of the cylindrical part)

The following is a list of numbered items comprised by the present invention:
1. Pumping unit for delivering a nebulised medically active aerosol for inhalation therapy, said unit comprising a riser pipe (2), a hollow cylindrical part (3) having an interior space configured to receive an upstream end portion (21) of said riser pipe (2), said cylindrical part (3) being moveable on the riser pipe (2), wherein the cylindrical part and the riser pipe form a pumping chamber having a variable volume, wherein a gap (4) is present between a lateral outside surface (22) of the upstream end portion (21) of said riser pipe (2) and a lateral internal surface (31) of said cylindrical part (3), wherein a seal (1) is present between the outside surface (22) of said riser pipe (2) and the internal surface (31) of said cylindrical part (3), thus physically bridging said gap (4), characterized in that the seal (1) has a cap-like shape configured to fit around the upstream end portion (21) of the riser pipe (2), having at least one opening (11) for providing a fluid connection between an interior space of said riser pipe (2) and said pumping chamber, and in that at an interior surface (14) of the seal (1), at least one interior protrusion (16) is present which is configured to be received by at least one respective recess (23) of the riser pipe (2).
2. Pumping unit according to item 1, wherein the at least one protrusion (16) is present (i) as an annular protrusion, or (ii) as a discontinuous protrusion.
3. Pumping unit according to item 1 or 2, wherein the interior protrusion (16) is spaced apart from an upstream end (12) as well as a downstream end (13) of the seal (1).
4. Pumping unit according to any of items 1 to 3, wherein in a cross section along the longitudinal axis (L), the contour of the interior protrusion (16) has a concave shape, or a polygonal shape, with or without undercut.
5. Pumping unit according to any of the preceding items, wherein the seal (1) has at its exterior surface (15) an annular exterior protrusion (17).
6. Pumping unit according to item 5, wherein said exterior protrusion (17) has, viewed in upstream direction along a longitudinal axis (L), a cross section which has a diameter increasing portion (17A), followed by a diameter reducing portion (17B).
7. Pumping unit according to item 6, wherein, in a cross-sectional view along the longitudinal axis (L), both portions (17A, 17B) have sections with linear contours, and/or wherein the angle between both contours of the sections of said portions (17A, 17B) is a right angle.
8. Pumping unit according to item 7, wherein the angle of the linear section of the diameter increasing portion (17A) is 20 degrees with respect to the longitudinal axis (L).
9. Pumping unit according to any of items 5 to 8, wherein a region (18) upstream of the exterior protrusion (17) has a diameter (D1′) which is smaller than a nominal diameter (D1) of the seal (1).
10. Pumping unit according to any of items 5 to 9, wherein the largest diameter (19) of the exterior protrusion (17) lies within a plane (P) which is perpendicular to the longitudinal axis (L), and which crosses the interior protrusion (16).
11. Pumping unit according to any of the preceding items, wherein the riser pipe (2) has a nominal outer diameter (D2), and wherein the riser pipe's (2) upstream end portion (21) has a reduced diameter (D2′).
12. Pumping unit according to item 11, wherein the seal (1) has at its exterior surface (15) an annular exterior protrusion (17) as defined in any of claims 5 to 10, and wherein only the region of the seal (1) where the exterior protrusion (17) is present exceeds said nominal outer diameter (D2) of the riser pipe (2).
13. Pumping unit according to item 11 or 12, wherein the diameter of the seal (1) at its downstream end (13) matches said nominal outer diameter (D2).
14. Pumping unit according to any of items 5 to 13, wherein in a non-assembled state the largest diameter (19) of the exterior protrusion (17) exceeds the inner diameter (D3) of the cylindrical part (3), such as to be able to physically close the gap (4) between riser pipe (2) and cylindrical part (3) in an assembled state.
15. Pumping unit according to any of the preceding items, wherein the shore hardness of the seal (1) is adjusted to the pressure that the pumping chamber can be exposed to, and to the width of the gap (4), such that, when exposed to said pressure, a tight seal is provided, while elastic deformation of the same is allowed, but creeping of the exterior protrusion (17) is inhibited.

1. An inhalation device for delivering a nebulised medically active aerosol for inhalation therapy comprising a pumping unit, said pumping unit comprising a riser pipe, a hollow cylindrical part having an interior space configured to receive an upstream end portion of said riser pipe, said cylindrical part being moveable on the riser pipe, wherein the cylindrical part and the riser pipe form a pumping chamber having a variable volume, wherein a gap is present between a lateral outside surface of the upstream end portion of said riser pipe and a lateral internal surface of said cylindrical part, wherein a seal is present between the outside surface of said riser pipe and the internal surface of said cylindrical part, thus physically bridging said gap, and wherein the seal has a cap-like shape configured to fit around the upstream end portion of the riser pipe, having at least one opening for providing a fluid connection between an interior space of said riser pipe and said pumping chamber, and wherein at an interior surface of the seal, at least one interior protrusion is present which is configured to be received by at least one respective recess of the riser pipe.

2. Inhalation device according to claim 1, wherein the at least one protrusion is present as an annular protrusion, or as a discontinuous protrusion.

3. Inhalation device according to claim 1, wherein the interior protrusion is spaced apart from an upstream end as well as a downstream end of the seal.

4. Inhalation device according to claim 1, wherein in a cross section along the longitudinal axis, the contour of the interior protrusion has a concave shape, or a polygonal shape, with or without undercut.

5. Inhalation device according to claim 1, wherein the seal has at its exterior surface an annular exterior protrusion.

6. Inhalation device according to claim 5, wherein said exterior protrusion has, viewed in upstream direction along a longitudinal axis, a cross section which has a diameter increasing portion, followed by a diameter reducing portion.

7. Inhalation device according to claim 6, wherein, in a cross-sectional view along the longitudinal axis, both portions have sections with linear contours, and/or wherein the angle between both contours of the sections of said portions is a right angle.

8. Inhalation device according to claim 7, wherein the angle of the linear section of the diameter increasing portion is 20 degrees with respect to the longitudinal axis.

9. Inhalation device according to claim 5, wherein a region upstream of the exterior protrusion has a diameter which is smaller than a nominal diameter of the seal.

10. Inhalation device according to claim 5, wherein the largest diameter of the exterior protrusion lies within a plane which is perpendicular to the longitudinal axis, and which crosses the interior protrusion.

11. Inhalation device according to claim 1, wherein the riser pipe has a nominal outer diameter, and wherein the riser pipe's upstream end portion has a reduced diameter.

12. Inhalation device according to claim 11, wherein the seal has at its exterior surface an annular exterior protrusion as defined in claim 5, and wherein only the region of the seal where the exterior protrusion is present exceeds said nominal outer diameter of the riser pipe.

13. Inhalation device according to claim 11, wherein the diameter of the seal at its downstream end matches said nominal outer diameter.

14. Inhalation device according to claim 5, wherein in a non-assembled state the largest diameter of the exterior protrusion exceeds the inner diameter of the cylindrical part, such as to be able to physically close the gap between riser pipe and cylindrical part in an assembled state.

15. Inhalation device according to claim 1, wherein the shore hardness of the seal is adjusted to the pressure that the pumping chamber can be exposed to, and to the width of the gap, such that, when exposed to said pressure, a tight seal is provided, while elastic deformation of the same is allowed, but creeping of the exterior protrusion is inhibited.
